SEQUENCE LISTING (6 sequences)

SEQ ID NO: 1 - protein, 10 aa, Artificial Sequence
  synthetic peptide
SARWGPIMPW

SEQ ID NO: 2 - protein, 12 aa, Artificial Sequence
  synthetic sequence
CSARWGPIMPWC

SEQ ID NO: 3 - protein, 17 aa, Artificial Sequence
  synthetic peptide
CHPGDKQEDPNCLQADK

SEQ ID NO: 4 - protein, 12 aa, Artificial Sequence
  synthetic peptide
CEGLPADWAAAC

SEQ ID NO: 5 - protein, 12 aa, Artificial Sequence
  synthetic peptide
CSARWGPTMPWC

SEQ ID NO: 6 - protein, 12 aa, Artificial Sequence
  synthetic peptide
CRRGSRASGAHC

(19) United States
(12) Patent Application Publication
(10) Pub. No.: US 2021/0198319 A1
(43) Pub. Date: Jul. 1, 2021

(54) CYTOTOXIC PEPTIDES AND CONJUGATES THEREOF

(71) Applicant: AEBI LTD., Ness Ziona (IL)

(72) Inventors: Ilan MORAD, Ness Ziona (IL); Hanan ITZHAKI, Ness Ziona (IL)

(21) Appl. No.: 16/650,557

(22) PCT Filed: Sep. 20, 2018
(86) PCT No.: PCT/IL2018/051063

§ 371 Date: Mar. 25, 2020

Related U.S. Application Data
(60) Provisional application No. 62/563,663, filed on Sep. 27, 2017.

(51) Int. Cl. C07K 7/64; A61P 35/00; A61K 45/06

(52) U.S. Cl. CPC C07K 7/64; A61K 38/00; A61K 45/06; A61P 35/00

(57) ABSTRACT

The present invention provides cytotoxic peptides, analogs thereof and fragments thereof, conjugates of said peptides, analogs or fragments as well as compositions comprising same. In addition, uses of these compounds and compositions in treating cancer are provided.

### FIELD OF THE INVENTION

The present invention relates to cytotoxic peptides and conjugates thereof and to their compositions and uses.

### BACKGROUND OF THE INVENTION

Around the world, tremendous resources are being invested in prevention, diagnosis, and treatment of cancer which is one of the major causes of death in Europe and North America. Discovery and development of anticancer agents are the key focus of several pharmaceutical companies as well as nonprofit government and non-government organizations.

The discovery and development of anticancer drugs, especially cytotoxic agents, differs significantly from the drug development process for any other indication. The unique challenges and opportunities in working with these agents are reflected in each stage of the drug development process.

Although there is a growing number of anticancer compounds used in clinic, there is an unmet need for development of additional cytotoxic compounds with reduced adverse side effects that may be used in cancer treatment and in particular in targeted cancer therapy.

WO2018/061004 discloses constructs comprising toxins and peptides targeting to extracellular tumor antigens covalently connected through different scaffolds.

It would be advantageous to have peptides that are cytotoxic only when introduced into cells by targeting moieties that can deliver these agents specifically to the target cancer cells.

### SUMMARY OF THE INVENTION

The present invention is based on the surprising discovery of a novel peptide that was shown to possess cytotoxicity toward tumor cells, alone, as a conjugate or as part of a construct with other moieties, such as targeting peptides. The present invention thus provides cytotoxic peptides based on the discovered sequence, analogs, active fragments and conjugates thereof.

According to one aspect, the present invention provides a peptide comprising the amino acid sequence SARWGPIMPW (SEQ ID NO: 1), or an analog thereof, wherein the peptide or its analog consists of 10 to 30 amino acids. According to some embodiments, the peptide comprises amino acid sequence CSARWGPIMPWC (SEQ ID NO: 2). According to yet another embodiment, the peptide is selected from SEQ ID NO: 1 and SEQ ID NO: 2.

According to some specific embodiments, the peptide is cyclized.

According to some embodiments, the peptide analog of a sequence selected from SEQ ID NO: 1 and SEQ ID NO: 2, has at least 70% at least 80% or at least 90% sequence identity to said sequences.

According to some embodiments, the peptide analog comprises 1-4 modifications to the sequence of SEQ ID NOs: 1 or 2. According to some embodiments, the modifications are selected from addition of at least one amino acid, deletion of at least one amino acid, substitution of at least one amino acid, modification of carboxy or amino terminus of the peptide, creation of cyclization or modification of cyclization type, and combinations thereof. According to some embodiments, the peptide analog comprises 1-2 modifications relative to the parent peptide. According to some embodiments, the analog is a conservative analog, comprising 1-4 conservative substitutions of amino acids or conservative modification of amino or carboxy terminus of the patent peptide, or combinations thereof. According to other embodiments, conservative analog comprises 1, 2, 3, or 4 conservative substitutions within the amino acids SEQ ID NO: 2. According to some embodiments, the analog does not comprise substitution of the Isoleucine (Ile) residue. According to other embodiments, substitution of the Ile residue is to an amino acid residue other than Threonine (Thr).

According to some embodiments the peptide analog is a fragment of the peptide of SEQ ID NO: 1 or SEQ ID NO: 2 wherein 1-4 amino acids were deleted from said sequences. According to some embodiments, the fragment comprises from 7 to 11 consecutive amino acids of SEQ ID NO: 1 or 2 or of an analog of said sequences.

According to some embodiments, the peptide, peptide analog or fragment of the present invention is cyclic. According to some embodiments, cyclization is through a disulfide bond formed between the side chains of two cysteine residues of the sequence.

According to other embodiments of the present invention, the peptide, analog or fragment specifically binds to eukaryotic Elongation Factor 2 (eEF2). According to some embodiments, the eEF2 is human eEF2. According to some embodiments, the binding of the peptide, analog or fragment of the present invention to human eEF2 enhances its activity.

According to some embodiments, the present invention provides a cyclic peptide consisting of SEQ ID NO: 2, wherein the peptide enhances eEF2 activity.

According to some embodiments, the peptide, analog or fragment of the present invention is cytotoxic to mammalian cells. Thus according to certain embodiments, the peptide, analog or fragment of the present invention is for use in inducing cell death of target cells. According to certain embodiments, the target cells are cancer cells.

According to another aspect, the present invention provides a conjugate comprising at least one copy of a peptide, analog or fragment of the present invention. According to certain embodiments, the conjugate comprises two or more copies of the peptide, analog or fragment of the present invention. According some embodiments, the conjugate comprises amino acid sequence SEQ ID NO: 2. According to other embodiments, the conjugate comprises a cyclic peptide. According to some embodiments, the conjugate further comprises at least one cancer-targeting molecule. According to other embodiments, the conjugate comprises two or more copies of a cancer-targeting molecule. According to some embodiments, the conjugate comprises two or more different cancer targeting molecules. According to certain embodiments, the cancer-targeting molecule binds specifically to a cell surface receptor selected from EGFR, PD-L1, HER2, androgen receptor, benzodiazepine receptor, Cadherin, CXCR4, CTLA-4, CD2, CD19, endothelin receptor, ERBB4, FGFR, folate receptor, HER4, HGFR, Mucin 1, OGFR, PD-1, PD-L2, PDGFR, and VEGFR. According to some specific embodiments, the cancer-targeting molecule is a peptide. According to other embodiments, the cancer-targeting molecule is a polypeptide or protein. According to some specific embodiments, the polypeptide is an antibody or a fragment thereof. According to certain embodiments, the cancer-targeting molecule that targets EGFR is a peptide comprising amino acid sequence SEQ ID NO: 3, an analog or functional fragment thereof. According to other embodiments, the cancer-targeting molecule that targets PD-L1 is a peptide comprising amino acid sequence SEQ ID NO: 4, an analog or functional fragment thereof. According to some embodiment, the conjugate comprises multiple copies of a peptide comprising SEQ ID NO: 2, multiple copies of a peptide comprising SEQ ID NO: 3 and multiple copies of a peptide comprising SEQ ID NO: 4. According to some embodiments, the conjugate comprises a carrier, a spacer or a scaffold. According to particular embodiments, the peptides are bound to the carrier or scaffold. According to some embodiment, the conjugate further comprises a different toxin.

According to a further aspect, the present invention provides pharmaceutical compositions comprising the peptide, analog or fragment of the present invention or the conjugate of the present invention, and a pharmaceutically acceptable carrier. According to some embodiments, the pharmaceutical composition comprises a cyclic peptide comprising SEQ ID NO: 2. According other embodiments, the pharmaceutical composition comprises a conjugate of a cyclic peptide comprising amino acid sequence SEQ ID NO: 2. According to some such embodiments, the pharmaceutical composition comprises a plurality of peptides, analogs, fragments and/or conjugates of the present invention. According to some embodiments, the pharmaceutical composition is for use in killing a specific population of cells. According to some embodiments, the pharmaceutical composition is for use in treating cancer.

According to another aspect, the present invention provides a method of treating cancer in a subject in need thereof comprising administering to said subject a pharmaceutical composition of the present invention. According to some embodiments, the method of treating cancer in a subject in need thereof comprises administering to said subject a therapeutically effective amount of a peptide, analog, fragment or conjugate of the present invention. According to some embodiment, the pharmaceutical composition is administered as part of a treatment regimen in combination with at least one additional anti-cancer agent.

According to a further aspect, the present invention provides a polynucleotide sequence encoding the peptide, analog, fragment or conjugate of the present invention.

According to certain aspects, the present invention provides a nucleic acid construct comprising the polynucleotide of the present invention operably linked to a promoter.

According to a further embodiment, the present invention provides a vector comprising at least one polynucleotide or at least one nucleic acid construct of the present invention.

According to yet another aspect, the present invention provides a cell comprising the polynucleotide or the nucleic acid construct of the present invention. According to one embodiment, the cell expresses the peptide, analog or fragment of the present invention.

### BRIEF DESCRIPTION OF THE FIGURES

FIG. 1 shows the effect of treatment of A-549 human lung cancer cells with following constructs: PEG-E13.3-(PD-L1-GR)-GW2, PEG-E13.3-(PD-L1-GR)-(TB-GW2) and PEG-E13.3-(PD-L1-GR)-(TB-GW), at different concentrations. The pictures were taken after 48 hour of incubation with Test Items. FIG. 1A show the cells at T=0; FIG. 1B—negative control (PBS); FIGS. 1C-1E—the construct PEG-E13.3-(PD-L1-GR)-GW2 comprising the cyclic peptide of SEQ ID NO: 2 and targeting peptides to PD-L1 (SEQ ID NO: 4) and to EGFR (SEQ ID NO: 3), at concentrations 0.3, 1 and 3 μM, respectively; FIGS. 1F-1H—PEG-E13.3-(PD-L1-GR)-(TB-GW2) comprising the toxins of SEQ ID NO: 2 and SEQ ID NO: 6, and targeting peptides to PD-L1 (SEQ ID NO: 4) and to EGFR(SEQ ID NO: 3), at concentrations 0.3, 1 and 3 μM, respectively; and FIG. 11—PEG-E13.3-(PD-L1-GR)-(TB-GW) comprising the toxin of SEQ ID NO: 6, and targeting peptides to PD-L1 (SEQ ID NO: 4) and to EGFR (SEQ ID NO: 3), at 3 μM.

### DETAILED DESCRIPTION OF THE INVENTION

The present invention provides peptides comprising amino acid sequence SARWGPIMPW (SEQ ID NO: 1), analogs thereof or active fragments of said peptide or said analogs that retains its cytotoxic activity. Some of the peptides, analogs and fragments of the present invention are agonists of human eukaryotic elongation factor 2 (eEF2). It has been unexpectedly found that a construct comprising peptides of the present invention, conjugated with cancer targeting molecules was efficient in killing cancer cell.

According to one aspect, the present invention provides a peptide comprising amino acid sequence SEQ ID NO: 1. According to another embodiment, the present invention provides a peptide consisting of SEQ ID NO: 1. According to certain embodiments, the present invention provides a peptide comprising amino acid sequence SEQ ID NO: 2. According to other embodiments, the present invention provides a peptide consisting of SEQ ID NO: 2.

The term “peptide” refers to a short chain of amino acid residues linked by peptide bonds, i.e., a covalent bond formed between the carboxyl group of one amino acid and an amino group of an adjacent amino acid. The term “peptide” typically refers to short sequences having up to 50 amino acids. A chain of amino acids monomers longer than 50 amino acid is referred as a “polypeptide”. Such polypeptides, when having more than 50 amino acid residues, can also be classified as proteins.

According to some embodiments, the peptide, peptide analog or fragment of the present invention consists of 7 to 35 amino acids. According to another embodiment, the peptide, peptide analog or fragment of the present invention consists of 8 to 30 or 10 to 28 amino acids. According to other embodiments, each peptide consists of 10 to 25 amino acids. According to yet other embodiments, each peptide consists of 12 to 20 amino acids. According to some embodiments, the peptide consists of 8, 9, 10, 11, 12, 13, 14, 15, 16, 17, 18, 19 or 20 amino acids. Each possibility represents a separate embodiment of the invention. According to some embodiments, the peptide comprises amino acid sequence selected from SEQ ID NO: 1 and consists of 10 to 30 amino acids. According to some embodiments, the peptide comprises amino acid sequence of SEQ ID NO: 2 and consists of 10 to 30 amino acids.

According to any one of the above embodiments, the peptide is a cyclic peptide. Thus, in one embodiment, the present invention provides a cyclic peptide comprising SEQ ID NO: 2. According to another embodiment, the present invention provides a cyclic peptide consisting of SEQ ID NO: 2. According to a further embodiment, the present invention provides a cyclic peptide comprising SEQ ID NO: 1. According to yet another embodiment, the invention provides a cyclic peptide consisting of SEQ ID NO: 1.

The term “cyclic peptide” refers to a peptide having an intramolecular bond between two non-adjacent amino acids. The cyclization can be effected through a covalent or non-covalent bond. Intramolecular bonds include, but are not limited to, end-to-tail, backbone-to-backbone, side-chain to backbone and side-chain to side-chain bridges. According to some embodiments, the cyclization occurs between the side chains of two cysteine residues of the peptide, analogs of fragments, to form a disulfide bridge. According to other embodiments, the cyclization occurs between the N-terminal and C-terminal amino acids. According to some embodiments, the cyclization is effected via a spacer. According to some embodiments, the cyclization of a peptide consisting of SEQ ID NO: 2 is via a disulfide bond formed between the side chains of two cysteine residues.

The term “peptide” encompasses also the term “peptide analog”. The term “peptide analog” and “analog” are used herein interchangeably and refer to a peptide which contains substitutions, rearrangements, deletions, additions and/or chemical modifications in the amino acid sequence of the original, parent peptide, and retains the functional properties of the original peptide. For the purpose of this invention, the analog has at least 70% identity to the original peptide.

Thus, in some embodiments, the present invention provides an analog of a peptide comprising SEQ ID NO: 2. According to one embodiment, the present invention provides an analog of a peptide consisting of SEQ ID NO: 2. According to any one of such embodiments, the analog has at least 70% sequence identity to a peptide comprising SEQ ID NO: 2. According to other embodiments, the analog has at least 70% sequence identity to a peptide consisting SEQ ID NO: 2. According to another embodiment, the peptide analog has at least 80%, at least 90% or at least 95% sequence identity. According to some embodiments, the analog is cyclic. According to one embodiment, the present invention provides an analog of a peptide comprising amino acid sequence SEQ ID NO: 1. An analog of the peptide of SEQ ID NO: 2, should not comprises a substitution of the Isoleucine (Ile) residue into a Threonine (Thr) residue.

The substitutions of the amino acids may be conservative or non-conservative substitution. The non-conservative substitution encompasses substitution of one amino acid by any other amino acid. In one particular embodiment, the amino acid is substituted by a non-natural amino acid.

The term “amino acid” as used herein refers to an organic compound comprising both amine and carboxylic acid functional groups, which may be either a natural or non-natural amino acid. Non-limiting examples of non-natural amino acids include diaminopropionic acid (Dap), diaminobutyric acid (Dab), ornithine (Orn), aminoadipic acid, β-alanine, 1-naphthylalanine, 3-(1-naphthyl)alanine, 3-(2-naphthyl)alanine, γ-aminobutiric acid (GABA), 3-(aminomethyl) benzoic acid, p-ethynyl-phenylalanine, p-propargly-oxy-phenylalanine, m-ethynyl-phenylalanine, p-bromophenylalanine, p-iodophenylalanine, p-azidophenylalanine, p-acetylphenylalanine, azidonorleucine, 6-ethynyl-tryptophan, 5-ethynyl-tryptophan, 3-(6-chloroindolyl)alanine, 3-(6-bromoindolyl)alanine, 3-(5-bromoindolyl)alanine, azidohomoalanine, p-chlorophenylalanine, α-aminocaprylic acid, O-methyl-L-tyrosine, N-acetylgalactosamine-α-threonine, and N-acetylgalactosamine-α-serine. According to one embodiment, the substitution is substitution with a non-natural amino acid.

The substitution of one or more amino acids may be a conservative substitution, thus, the term peptide and analog encompass also the term conservative analog. The term “conservative analog” and “conservative peptide analog” are used herein interchangeably and refer to any peptide having an amino acid sequence substantially identical to one of the sequences specifically shown herein in which one or more residues have been conservatively substituted with a functionally similar residue and which displays the abilities as described herein.

Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. One of skill will recognize that individual substitutions, is a “conservatively modified variant” where the alteration results in the substitution of an amino acid with a chemically similar amino acid. Conservative substitution tables providing functionally similar amino acids are well known in the art. One typical non-limiting example of conservative substitution is provided below.

The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). In other embodiments, the conservative substitution encompass substitution with a chemically similar non-natural amino acid.

In some embodiments, the present invention provides a conservative analog of a peptide comprising SEQ ID NO: 2. According to one embodiment, the substitution is within the sequence SEQ ID NO: 2. In some embodiments, the present invention provides a conservative analog of a peptide consisting of SEQ ID NO: 2. According to any one of such embodiments, the analog has at least 70% sequence identity to the peptide comprising or consisting of SEQ ID NO: 2. According to another embodiment, the peptide analog has at least 80%, at least 90% or at least 95% identity. According to one embodiment, the analog has about 70% to about 95%, about 80% to about 90% or about 85% to about 95% sequence identity to the original peptide. According to some embodiments, the analog is cyclic. In some embodiments, the present invention provides a conservative analog of a peptide comprising SEQ ID NO: 1. According to one embodiment, the peptide analog has at least 80%, at least 90% or at least 95% identity to a peptide consisting of amino acid sequence SEQ ID NO: 1.

According to some embodiments, the analog of the present invention comprises amino acid sequence SEQ ID NO: 2 in which 1, 2, 3, or 4 substitutions were made. According to some embodiments, the conservative analog of the present invention comprises the amino acid sequence SEQ ID NO: 2 in which 1, 2, 3, or 4 conservative substitutions were made. According to another embodiment, the conservative analog consists of SEQ ID NO: 2 in which 1, 2 or 3 conservative substitution were made. Thus, the conservative analog consists of SEQ ID NO: 2 with 1, 2 or 3 conservative substitutions. According to a further embodiment, the conservative analog comprises 1, 2, 3, or 4 conservative substitutions of the amino acids within the amino acid sequence SEQ ID NO: 2. According to some embodiments, the analog is cyclic.

According to other embodiments, the analog of the present invention comprises amino acid sequence SEQ ID NO: 1 in which 1, 2, 3, or 4 substitutions were made. According to some embodiments, the conservative analog of the present invention comprises the amino acid sequence SEQ ID NO: 1 in which 1, 2, 3, or 4 conservative substitutions were made. According to another embodiment, the conservative analog consists of SEQ ID NO: 1 in which 1, 2 or 3 conservative substitution were made. Thus, the conservative analog consists of SEQ ID NO: 1 with 1, 2 or 3 conservative substitutions. According to some embodiments, the analog is cyclic.

The term “peptide” also encompasses also the term “peptide fragment”. The term “fragment” refers to a fragment of the original peptide or of an analog of the original peptide thereof wherein said fragment retains the activity of the original peptide or analog. Thus, the terms “fragment” and “active fragment” may be used interchangeably. According to one embodiment, the present invention provides a fragment of a peptide comprising amino acid SEQ ID NO: 2. According to another embodiment, the present invention provides a fragment of a peptide consisting of SEQ ID NO: 2. According to yet another embodiment, the present invention provides a fragment of an analog of a peptide comprising or consisting of SEQ ID NO: 2. According to some embodiments, the fragment consists of at least 6, at least 7, at least 8, at least 9, or at least 10 consecutive amino acids of SEQ ID NO: 2 or of an analog thereof. According to one embodiment, the fragment consists of 6 to 11, 7 to 10 or 8 to 9 of consecutive amino acids of SEQ ID NO: 2 or analog thereof. According to other embodiments, the present invention provides a fragment of a peptide comprising or consisting of amino acid SEQ ID NO: 1. According to yet another embodiment, the present invention provides a fragment of an analog of a peptide comprising or consisting of SEQ ID NO: 1.

According to any one of the aspects and embodiments of the invention, the terms “peptide comprising the amino acid sequence set forth in SEQ ID NO: X”, “peptide having SEQ ID NO: X”, “peptide comprising amino acid SEQ ID NO: X” and “peptide comprising SEQ ID NO: X” are used herein interchangeably. The terms “peptide consisting of the amino acid sequence set forth in SEQ ID NO: X”, “peptide of SEQ ID NO: X”, “peptide consisting of amino acid SEQ ID NO: X” and “peptide consisting of SEQ ID NO: X” are used herein interchangeably.

According to any one of the above embodiments, the peptide, analog, conservative analog and fragment of the present invention binds specifically to human eukaryotic Elongation Factor 2 (eEF2). The term “binds specifically” means that the molecule such as a peptide has a substantially greater affinity for their target entity such as antigen than their affinity to other related entities, and preferably do not cross-react with these related entities. According to one embodiment, binding of the peptide, analog, conservative analog or fragment to human eEF2 enhances the activity of human eEF2. According to one embodiment, the peptide comprising amino acid SEQ ID NO: 2 enhances human eEF2 activity. According to another embodiment, the peptide consisting of amino acid SEQ ID NO: 2 enhances human eEF2 activity. According to a further embodiment, the cyclic peptide comprising or consisting of amino acid SEQ ID NO: 2 enhances human eEF2 activity. According to another embodiment, the analog of the peptide comprising or consisting of amino acid SEQ ID NO: 2 enhances human eEF2 activity. According to another embodiment, the analog comprising or consisting of SEQ ID NO: 2 with one, two or three conservative substitutions enhances human eEF2 activity. According to another embodiment, the peptide selected from a peptide comprising or consisting amino acid sequence SEQ ID NO: 1, cyclic peptide comprising or consisting of amino acid SEQ ID NO: 1 and the analog of the peptide comprising or consisting of amino acid SEQ ID NO: 1 enhances human eEF2 activity.

The terms “enhances activity” and “increases activity” are used herein interchangeably. According to one embodiment, the enhanced activity means that the activity is from 10% to 100%, from 20% to 90%, from 30% to 80%, from 40% to 70% or from 50 to 60% higher than the activity of a native untreated eEF2. According to another embodiment, the enhanced activity comprises from 5% to 25%, from 25% to 50% from 50% to 75% or from 75% to 100% higher activity. According to a further embodiment, the enhanced activity comprises at least 1.5, at least 2, at least 2.5 at least 3, at least 5 or at least 10 folds higher activity than the activity of a native untreated eEF2 measured at the same conditions.

According to one embodiment, the present invention provides a cyclic peptide consisting of SEQ ID NO: 2 that enhances human eEF2 activity.

According to any one of the above embodiments, the peptide, analog, conservative analog or fragment of the present invention is a toxin.

As used herein, the term “toxin” refers to a substance, which is poisonous, harmful or cytotoxic to mammalian cells, such as human cells.

According to one embodiment, the peptide comprising amino acid sequence SEQ ID NO: 2 is a toxin. According to one embodiment, the peptide consisting of amino acid SEQ ID NO: 2 is a toxin. According to one embodiment, the peptide is a cyclic peptide. According to another embodiment, the analog of a peptide comprising or consisting of amino acid SEQ ID NO: 2, or the fragment of the peptide or of the analog is a toxin. According to another embodiment, the peptide selected from a peptide comprising or consisting amino acid sequence SEQ ID NO: 1, the cyclic peptide comprising or consisting of amino acid SEQ ID NO: 1 and the analog of the peptide comprising or consisting of amino acid SEQ ID NO: 1 analog is a toxin.

Thus, in one embodiment, the peptide, analog or fragment according to the present invention induces cell death. In another embodiment, the peptide, analog or fragment is for use in inducing cell death of target cells. According to one embodiment, the peptide comprising or consisting of SEQ ID NO: 2 is for use in inducing cell death of target cells. According to one embodiment, the analog of a peptide comprising or consisting of SEQ ID NO: 2, or the fragment of the peptide or of the analog is for use in inducing cell death of target cells. According to some embodiments, the target cells are cancer cells. According to another embodiment, the peptide is selected from a peptide comprising or consisting amino acid sequence SEQ ID NO: 1, the cyclic peptide comprising or consisting of amino acid SEQ ID NO: 1 and the analog of the peptide comprising or consisting of amino acid SEQ ID NO: 1 is for use in inducing cell death in target cells.

The terms “induce cell death” and “promote cell death” are used herein interchangeably and mean that the peptide, the analog or the fragment of the present invention can directly induce cell death, where cell death includes apoptosis and necrosis. The cell death may be caused due to interaction of the compound of the present invention with molecules expressed on the cell surface or with molecules located within the cell such as molecule located in the cytosol, bound to the inner side of the cell membrane, located in the organelles or present on the membrane of the organelles, either inner or outer part of it.

The term “cell death” as used herein encompasses both destruction and damage or impairment of cells. The term “cell death” encompasses cell ablation.

The cytotoxic activity of the peptide, analog or fragment of the present invention may be tested using any known method such as Alamar test.

The peptides of present invention may be produced by any method known in the art, including recombinant (for peptides consisting of genetically encoded amino acids) and synthetic methods. Synthetic methods include exclusive solid phase synthesis, partial solid phase synthesis, fragment condensation, or classical solution synthesis. Solid phase peptide synthesis procedures are well known to one skilled in the art. Synthetic methods to produce peptides include but are not limited to FMOC solid phase peptide synthesis described, for example in Fields G. B., Noble R., Int. J. Pept. Protein Res., 35: 161-214, 1990. Methods for synthesizing peptides on PEG are described for example in DeNardo et al. Ibid.

In some embodiments, synthetic peptides are purified by preparative high performance liquid chromatography and the peptide sequence is confirmed via amino acid sequencing by methods known to one skilled in the art.

In some embodiments, recombinant protein techniques, well known in the art, are used to generate peptides and peptide multimers (consisting of genetically encoded amino acids) of the present invention.

According to another aspect, the present invention provides a conjugate comprising at least one copy of the peptide, analog, conservative analog or fragment of the present invention.

The term “conjugate” refers to any compound formed from the joining together or binding of two or more molecules. In particular, the term conjugate encompasses a compound formed from binding of two or more copies of the peptide, analog or fragment of the present invention or a compound comprising said peptide, analog or fragment bound to another molecule. According to some embodiments, the peptide of the present invention within a conjugate appears as a moiety of the peptide. The moiety has the same function and efficacy as the parent peptide. The term “moiety” as used herein refers to a part of a molecule, which lacks one or more atom(s) compared to the corresponding molecule as a result of the binding. According to some embodiments, the conjugate comprises a moiety of the peptide, analog or fragment of the present invention.

According to some embodiments, the present invention provides a conjugate comprising at least one copy of a peptide comprising or consisting of an amino acid sequence selected from SEQ ID NO: 2 and SEQ ID NO: 1. According to some embodiments, the present invention provides a conjugate comprising at least one copy of a peptide comprising an amino acid sequence SEQ ID NO: 2. According to another embodiment, the present invention provides a conjugate comprising at least one copy of a peptide consisting of SEQ ID NO: 2. According to some such embodiments, the peptide is a cyclic peptide. According to other embodiments, the present invention provides a conjugate comprising at least one copy of an analog of a peptide comprising SEQ ID NO: 2. According to a further embodiment, the conjugate of comprising at least one copy of an analog of the peptide consisting of SEQ ID NO: 2. According to some embodiments, the analog is cyclic analog. According to other embodiments, the present invention provides a conjugate comprising at least one copy of a fragment of the peptide or of the analog of the present invention. According to some embodiments, the analog has one or two conservative substitutions in SEQ ID NO: 2. According to some embodiments, the conjugate comprises a moiety of the above peptides. According to some embodiments, the present invention provides a conjugate comprising at least one copy of a peptide comprising an amino acid sequence SEQ ID NO: 1, analog, or a fragment thereof.

According to some embodiments, the conjugate comprises two or more copies of the peptide, analog, conservative analog or fragment of the present invention bound directly. According to another embodiment, the conjugate comprises two or more copies of the peptide, analog, conservative analog or fragment of the present invention bound via a spacer or a carrier. According to one embodiment, the conjugate comprises a plurality of copies of the peptide, analog, conservative analog or fragments of the present invention.

According to another embodiment, the present invention provides a conjugate comprising at least one copy of the peptide, analog, conservative analog or fragment of the present invention bound to another molecule, directly or via a spacer or carrier. According to some embodiments, the molecule that may be bound to the peptide, analog, conservative analog or fragment of the present invention can be any molecule. According to some embodiments, the molecule is selected from an active agent, cell-targeting molecule, an extracellular cancer-targeting molecule, a carrier, a toxin, a permeability-enhancing moiety, an anti-cancer agent and any combination thereof.

As used herein, the term “cell-targeting”, when referring to a molecule, indicates that the molecule provides specific cell targeting. In some embodiments, the cell is cancer cell. Thus, in some embodiment, the cell targeting have the meaning of cancer targeting. In particular, a cell-targeting molecule, such as a peptide or antibody, specifically recognizes and binds a cell target on cancer cells. By virtue of its binding, the cancer-targeting moiety directs the cytotoxic peptides, analogs or fragments of the present invention to the cancerous tissue, to facilitate specific killing/inhibition of cancerous cells.

The terms “carrier” refers to any molecule that covalently binds or capable of binding to the at least two different peptides and/or toxins. Several possible binding arrangements are encompassed. According to one embodiment, one peptide and one toxin are bound via a carrier and the second peptide is bound directly to the first peptide or to the toxin. According to another embodiment, two peptides are bound via a carrier, and the toxin is bound to one of the peptides. According to a further embodiment, all peptides and toxin(s) are covalently bound to a carrier. According to some embodiments, the carrier is a scaffold.

According to any one of the above embodiment, the peptides and/or the toxin(s) are bound via a linker. The terms “linker” and “spacer” are used herein interchangeably and refer to any molecule that covalently binds and therefore linking two molecules. Non-limiting examples of the linker are amino acids, peptides, or any other organic substance that can be used to allow distance between two linked molecules.

According to some embodiments, the conjugate comprises at least one copy of the peptide of the present invention bound to at least one copy of a cell-targeting molecule. According to another embodiment, the peptide is bound to multiple copies of said cell-targeting molecule. According to certain embodiments, the at least one copy of the peptide of the present invention is bound to two or more different cell-targeting molecules. According to some embodiments, the at least one copy of the peptide of the present invention is bound to 2 to 10, 3 to 8, 4 to 6 different cell-targeting molecules. According to another embodiment, the peptide is bound to multiple copies of each one of two or more cell-targeting molecules. According to some embodiments, the cell-targeting molecules are cancer-targeting molecules. According to one embodiment, the peptide comprises SEQ ID NO: 2. According to another embodiment, the peptide consists of SEQ ID NO: 2. According to some embodiments, the peptide is a cyclic peptide. According to some embodiments, the cell-targeting molecule targets a cell surface receptor selected from EGFR, PD-L1, HER2, androgen receptor, benzodiazepine receptor, Cadherin, CXCR4, CTLA-4, CD2, CD19, endothelin receptor, ERBB4, FGFR, folate receptor, HER4, HGFR, Mucin 1, OGFR, PD-1, PD-L2, PDGFR, and VEGFR.

As used herein, the terms “target” and “cell target” refer to molecules found on cancer cells that may be a marker of cancer cell and may be involved in cancer cell growth, proliferation, survival and metastasis development. Particular examples of targets include cell-surface proteins, which upon binding to their counterparts, such as ligands, initiate a cascade that promotes tumor growth and development. A target according to the present invention is optionally highly expressed on cancer cells and not found, or found in substantially lower levels, on normal non-cancerous cells. The term “target” encompass therefore the term “extracellular tumor antigen”. The term “tumor antigen” or “extracellular tumor antigen” are used herein interchangeably and include both tumor associated antigens (TAAs) and tumor specific antigens (TSAs). A tumor-associated antigen means an antigen that is expressed on the surface of a tumor cell in higher amounts than is observed on normal cells or an antigen that is expressed on normal cells during fetal development. A tumor specific antigen is an antigen that is unique to tumor cells and is not expressed on normal cells. The term tumor antigen includes TAAs or TSAs that have been already identified and those that have yet to be identified and includes fragments, epitopes and any and all modifications to the tumor antigens.

As used herein, the term “cell-targeting”, indicates that the molecule such as a peptide provides cell-, tissue- or organ-specific targeting. In particular, a cell-targeting peptide specifically recognizes and binds a cell target on cancer cells. By virtue of its binding, the cell-targeting peptide directs the entire construct to the cancerous tissue, to facilitate specific killing/inhibition of cancerous cells Killing/inhibition of cancerous cells is typically affected via the toxin, e.g. the peptide, of the present invention.

### Constructs

According to some embodiments, the conjugate comprises at least one copy of the peptide of the present invention bound to at least one copy of at least two different peptides that bind specifically to at least two different extracellular tumor antigens (antigen-targeting peptides) to form a construct. According to some embodiments, the peptide of the present invention and the antigen-targeting peptides are covalently bound directly or through a carrier.

According to some embodiments of the invention, at least one of the antigen-targeting peptides binds specifically to an extracellular tumor antigen selected from human epidermal growth factor receptor (EGFR) and human Programmed death-ligand 1 (PD-L1). In certain embodiments, the another one of the at least two antigen-targeting peptides binds specifically to an extracellular tumor antigen selected from the group consisting of EGFR, PD-L1, HER2, androgen receptor, benzodiazepine receptor, Cadherin, CXCR4, CTLA-4, CD2, CD19, endothelin receptor, ERBB4, FGFR, folate receptor, HER4, HGFR, Mucin 1, OGFR, PD-1, PD-L2, PDGFR, and VEGFR.

According to any one of the above embodiments, the at least one copy of the peptide of the present invention and the antigen-targeting peptides are covalently bound to form a construct. According to some embodiments, the peptide(s) and the antigen-targeting peptides are bound through a carrier to form a construct. According to some embodiments, the construct comprises from 3 to 10 different peptides binding to different extracellular tumor antigens.

According to one embodiment, the construct comprises a plurality of copies of the peptides of the present invention comprising or consisting of amino acid sequence SEQ ID NO: 2 and a plurality of the peptides that bind specifically to EGFR. According to one embodiment, the peptide that bind specifically to EGFR comprises amino acid sequence in SEQ ID NO: 3 (CHPGDKQEDPNCLQADK) or being an analog thereof.

According to another embodiment, the construct comprises a plurality of copies of the peptide of the present invention comprising or consisting of amino acid sequence SEQ ID NO: 2 and a plurality of the peptides that bind specifically to PD-L1. According to one embodiment, the peptide that bind specifically to PD-L1 comprises amino acid sequence as set forth in SEQ ID NO: 4 (CEGLPADWAAAC) or being an analog thereof.

In certain embodiments, the present invention provides a construct comprising two or more different antigen-targeting peptides binding to at least two different extracellular tumor antigens, a plurality of the peptides of the present invention, wherein the peptides and the antigen-targeting peptides are covalently bound directly or through a carrier and wherein one of the antigen-targeting peptides binds specifically to EGFR and one of the peptides binds specifically to PD-L1. According to one embodiment, the antigen-targeting peptide(s) that binds specifically to EGFR is a peptide having SEQ ID NO: 3 or an analog thereof, and the antigen-targeting peptide(s) that binds specifically to PD-L1 is a peptide having SEQ ID NO: 4 or an analog thereof. According to some embodiments, the construct further comprises an additional toxin. According to some embodiment, the additional toxin is selected from eukaryotic elongation factor 2 enhancer, BIM-BH3 consisting of SEQ ID NO: 5, Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin. In one embodiment, the toxin that binds specifically to eukaryotic elongation factor 2 is a toxin comprising the amino acid sequence selected from SEQ ID NO: 5 (CSARWGPTMPWC), SEQ ID NO: 6 (CRRGSRASGAHC), or an analog thereof. According to some embodiments, the construct comprises 2 to 10 different toxins. According to certain embodiments, the construct further comprises a toxin having SEQ ID NO: 5 and a toxin having SEQ ID NO: 6.

According to some embodiments, the construct comprises from 2 to 100 copies of the peptide of the present invention. According to another embodiment, the construct comprises from 2 to 100 copies of a peptide comprising or consisting of amino acid sequence SEQ ID NO: 3. According to a further embodiment, the construct comprises from 2 to 100 copies of a peptide comprising or consisting of amino acid sequence SEQ ID NO: 4.

According to some embodiments, the construct comprises multiple copies of the peptide of the present invention. According to one embodiment, the peptide comprises amino acid sequence SEQ ID NO: 2. According to another embodiment, the peptide consists of amino acid sequence SEQ ID NO: 2. According to some other embodiments, the construct comprises multiple copies of the peptide having the SEQ ID NO: 3, analog or fragment thereof and multiple copies of the peptide having the SEQ ID NO: 4, analog or fragment thereof. According to some embodiments, the construct comprises from 2 to 100, 3 to 90, 4 to 60, 5 to 50, 6 to 40, 7 to 35, 8 to 30, 9 to 25 or 10 to 20 copies of the peptide having SEQ ID NO: 2, analog or fragment thereof. According to some embodiments, the construct comprises from comprises from 2 to 100, 3 to 90, 4 to 60, 5 to 50, 6 to 40, 7 to 35, 8 to 30, 9 to 25 or 10 to 20 copies of the peptides comprising amino acid sequence SEQ ID NO: 3 and 4. According to some embodiments, the construct comprises from 7 to 56, from 14 to 48, from 21 to 42 from 28 to 35, or from 7 to 21 copies of the peptides comprising amino acid sequences SEQ ID NO: 2, 3 and 4. According to some embodiments, the construct comprises from 7 to 56, from 14 to 48, from 21 to 42 from 28 to 35, or from 7 to 21 copies of the peptides consisting of amino acid sequences SEQ ID NO: 2, 3 and 4.

According to any one of the above embodiments, at least one copy of the peptide of the present invention and at least one copy of the antigen-targeting peptides are covalently bound through a carrier.

According to some embodiments, the scaffold is a peptidic scaffold. According to other embodiments, the peptidic scaffold connects the peptides to each other on a single location in the scaffold, or to a different location on a scaffold. Each possibility represents a separate embodiment of the invention. According to some embodiments, the scaffold comprises at least one Lysine (Lys) residue. According to other embodiments, the scaffold comprises at least three Lys residues. According to further embodiments, the at least three Lys residues are connected together by amide bonds to form a branched multimeric scaffold. According to some embodiments, at least one amide bond is formed between the epsilon amine of a Lys residue and the carboxy group of another Lys residue.

According to a particular embodiment, the construct comprises a molecule according to one of the schemes presented below:

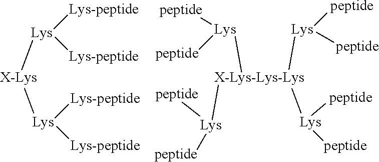

wherein X represents the peptide's and/or the toxin's C-terminal selected from carboxy acid, amide or alcohol group and optionally a linker or spacer, and peptide denotes a peptide according to the present invention, e.g. having 7-20 amino acids capable of binding to a cell-target. Each possibility represents a separate embodiment of the present invention.

According to some specific embodiments, at least one of the peptides of the present invention and/or the toxin(s) is present in multiple copies. According to some embodiments, the multiple copies are linked thereby forming a multi-target peptide multimer. According to some embodiments, the peptide and/or the toxin(s) copies are linked through a linker. According to other embodiments, the peptides and/or the toxin(s) copies are linked directly. According to a further embodiments, the multimer comprises copies linked both directly and via a linker.

According to some embodiments, the construct comprises a peptide multimer comprising a plurality of cell-targeting peptides arranged in an alternating sequential polymeric structure B(X₁X₂X₃ . . . Xₘ)ₙB or in a block copolymer structure B(X₁)_{nZ}(X₂)_{nZ}(X₃)ₙZ . . . (Xₘ)ₙ, wherein B is an optional sequence of 1-10 amino acid residues; n is at each occurrence independently an integer of 2-50; m is an integer of 3-50; each of X₁, X₂ . . . Xₘ is an identical or different peptide consisting of 5-30 amino acid residues; Z at each occurrence is a bond or a spacer of 1-4 amino acid residues. According to particular embodiments, n is at each occurrence independently an integer of 2-10; m is an integer of 3-10; each of X₁, X₂ . . . Xₘ is an identical or different peptide consisting of 7-20 amino acid residues; Z at each occurrence is a bond or a spacer of 1-4 amino acid residues. Each possibility represents a separate embodiment of the present invention.

According to some embodiments, the peptide multimer comprises 2-8 different or identical peptides. According to a particular embodiment, the peptide multimer comprises 4-10 copies of a single peptide sequence. According to yet other embodiments, the peptide multimer consists of 2-10, 3-9, 4-8, or 10-100 different or identical peptides. Each possibility represents a separate embodiment of the present invention.

According to other embodiments, the scaffold comprises or formed from a polyethylene glycol (PEG) molecule(s) or a modified PEG molecule(s). According to certain embodiments, the scaffold comprises a branched PEG molecule. According to some embodiments, the branched molecule comprises at least two sites available to bind a peptide of the present invention. According to other embodiments, the scaffold comprises from 2 to 100, 3 to 90, 4 to 60, 5 to 50, 6 to 40, 7 to 35, 8 to 30, 9 to 25 or 10 to 20, or 2 to 50 sites available to bind a peptide. According to one embodiment, the construct comprises from 7 to 56, from 14 to 48, from 21 to 42 from 28 to 35, from 7 to 21 sites available to bind a peptide. According to certain embodiment, the scaffold comprises 8 or 56 sites available to bind a peptide. According to further embodiments, the scaffold comprises 42 or 49 to 56 sites available for binding a peptide.

According to some embodiments, the PEG molecule is a branched molecule, comprising at least two separate connections to a peptide. According to some embodiments, the PEG has 8 binding sites. According to other embodiments, the PEG is bound to additional PEG molecules. According to certain embodiments, multiple PEG molecules are bound to provide a multi-armed PEG molecule. According to some embodiments, 8-armed PEG molecules are abound to one central 8-armed PEG molecule to provide one PEG molecules with 56 sites capable of binding the peptides of the present invention or the cell-targeting molecules such as cancer-targeting peptides. According certain embodiments, the peptides are connected to the PEG scaffold through amide bonds formed between amino groups of an NH₂—PEG molecule. According to yet other embodiments, at least one peptide is connected to PEG scaffold though a Lys residue.

According to some embodiment, the scaffold is a polyamidoamine multibranched scaffold.

According to any one of the above embodiments, the conjugate may comprise a permeability-enhancing moiety. The term “permeability-enhancing moiety” as used herein refers to any moiety known in the art capable of facilitating or enhancing, actively or passively, the permeability of the compound through body barriers or into the cells. According so one embodiment, any such may be used for conjugation with the peptide, analog or fragment of the present invention. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to a one embodiment, the hydrophobic moiety is a lipid moiety or an amino acid moiety.

The permeability-enhancing moiety may be connected to directly or through a spacer.

According to another aspect, the present invention provides a composition comprising the peptide, analog, conservative analog, fragment or conjugate of the present invention. According to one embodiment, the composition is a pharmaceutical composition. Thus, in some embodiments, the present invention provides a pharmaceutical composition comprising the peptide, analog, conservative analog, fragment or conjugate of the present invention, and a pharmaceutically acceptable carrier.

According to one embodiment, the pharmaceutical composition comprises a peptide comprising amino acid sequence SEQ ID NO: 2. According to another embodiment, the pharmaceutical composition comprises an analog or a conservative analog of a peptide comprising or consisting of SEQ ID NO: 2. According to a further embodiment, the pharmaceutical composition comprises a fragment of the peptide or of the analog. According to some embodiments, the pharmaceutical composition comprises the peptide consisting of SEQ ID NO: 2. According to some embodiments, the peptide, analog or fragment is cyclic. According to certain embodiments, the peptide, analog, conservative analog or fragment enhances human eEF2 activity.

According to another embodiment, the pharmaceutical composition comprises a peptide comprising amino acid sequence SEQ ID NO: 1. According to another embodiment, the pharmaceutical composition comprises an analog or a conservative analog of a peptide comprising or consisting of SEQ ID NO: 1. According to a further embodiment, the pharmaceutical composition comprises a fragment of the peptide comprising amino acid sequence SEQ ID NO: 1 or of the analog. According to some embodiments, the pharmaceutical composition comprises the peptide consisting of SEQ ID NO: 1. According to some embodiments, the peptide, analog or fragment is cyclic. According to certain embodiments, the peptide, analog, conservative analog or fragment enhances human eEF2 activity.

In one embodiment, the present invention provides a pharmaceutical composition comprising a cyclic peptide comprising amino acid sequence SEQ ID NO: 2. In another embodiment, the present invention provides a pharmaceutical composition comprising a cyclic peptide consisting of SEQ ID NO: 2. According to another embodiment, the present invention provides a pharmaceutical composition comprising a cyclic peptide comprising or consisting of SEQ ID NO: 1.

According to another embodiment, the present invention provides a pharmaceutical composition comprising a conjugate of the peptide, analog or fragment of the present invention. According to one embodiment, the pharmaceutical composition comprises a conjugate of a peptide comprising the amino acid sequence SEQ ID NO: 2. According to another embodiment, the pharmaceutical composition comprises a conjugate of an analog or a conservative analog of a peptide comprising or consisting of SEQ ID NO: 2. According to a further embodiment, the pharmaceutical composition comprises a conjugate of a fragment of the peptide or the analog as described above. According to some embodiments, the pharmaceutical composition comprises the conjugate of the peptide consisting of SEQ ID NO: 2. According to some embodiments, the peptide, analog or fragment is cyclic. According to certain embodiments, the peptide, analog, conservative analog or fragment enhances human eEF2 activity. According to one embodiment, the conjugate comprises two or more copies of the peptide, analog, conservative analog or fragment of the present invention bound directly or via a spacer or a carrier. According to one embodiment, the conjugate comprises the peptide, analog conservative analog or fragment bound to another molecule, directly or via a spacer or carrier. According to a further embodiment, the conjugate comprises the peptide, analog conservative analog or fragment bound to one or more cell-targeting molecules. According to some embodiments, the cell-targeting molecule target a cell surface receptor selected from EGFR, PD-L1, HER2, androgen receptor, benzodiazepine receptor, Cadherin, CXCR4, CTLA-4, CD2, CD19, endothelin receptor, ERBB4, FGFR, folate receptor, HER4, HGFR, Mucin 1, OGFR, PD-1, PD-L2, PDGFR, and VEGFR. According to a further embodiment, the pharmaceutical composition comprises a conjugate of a fragment of the peptide or the analog as described above. According to other embodiments, the pharmaceutical composition comprises the conjugate of the peptide comprising amino acid sequence SEQ ID NO: 1. According to certain embodiments, the pharmaceutical composition comprises the conjugate of peptide consisting of amino acid sequence SEQ ID NO: 1.

According to one embodiment, the pharmaceutical composition comprises a conjugate comprising at least one copy of the peptide comprising or consisting of SEQ ID NO: 2 bound to one or more cell-targeting molecules selected from a molecule targeting a cell surface receptor selected from EGFR, PD-L1, HER2, androgen receptor, benzodiazepine receptor, Cadherin, CXCR4, CTLA-4, CD2, CD19, endothelin receptor, ERBB4, FGFR, folate receptor, HER4, HGFR, Mucin 1, OGFR, PD-1, PD-L2, PDGFR, and VEGFR. According to one embodiment, the cell-targeting molecule is a cancer-targeting molecule.

According to one embodiment, the cell-targeting molecule is a peptide that bind specifically to EGFR. According to one embodiment, the EGFR-binding peptide comprises amino acid sequence in SEQ ID NO: 3.

According to one embodiment, the cell-targeting molecule is a peptide that bind specifically to PD-L1. According to one embodiment, the PD-L1-binding peptide comprises amino acid sequence in SEQ ID NO: 3.

According to one embodiment, the pharmaceutical composition comprises a conjugate comprising at least one copy of the peptide comprising amino acid sequence SEQ ID NO: 2 bound at least one copy of a peptide comprising amino acid sequence SEQ ID NO: 3 and to at least one copy of a peptide comprising amino acid sequence SEQ ID NO: 4. According to one embodiment, the peptides are bound via a scaffold to form a construct. According to another embodiment, the construct comprises from 2 to 100 copies of peptides comprising amino acid sequences SEQ ID NO: 2, 3 and 4.

According to some embodiments, the pharmaceutical composition of the present is for use in treating a cell proliferative disease. According to one embodiment, the cell proliferative disease is cancer. According to one embodiment, the pharmaceutical composition comprising a peptide comprising SEQ ID NO: 2 is for use in treating cancer. According to another embodiment, the pharmaceutical composition comprising a peptide consisting of SEQ ID NO: 2 is for use in treating cancer. According to yet another embodiment, the pharmaceutical composition comprising a conjugate of a peptide comprising or consisting of SEQ ID NO: 2 is for use in treating cancer. According to some embodiments, the pharmaceutical composition comprising an analog such as a conservative analog of a peptide comprising or consisting of SEQ ID NO: 2 is for use in treating cancer. According to certain embodiments, the pharmaceutical composition comprising a conjugate of an analog of a peptide comprising or consisting of SEQ ID NO: 2 is for use in treating cancer. According to any one of the above embodiments, the peptide, the analog or the fragment is cyclic peptide, analog or fragment.

According to one embodiment, the pharmaceutical composition comprising a peptide comprising or consisting of amino acid sequence SEQ ID NO: 1, is for use in treating cancer. According to yet another embodiment, the pharmaceutical composition comprising a conjugate of a peptide comprising or consisting of SEQ ID NO: 1 or an analog thereof is for use in treating cancer. According to any one of the above embodiments, the peptide, the analog or the fragment is cyclic peptide, analog or fragment.

The term “pharmaceutical composition” as used herein refers to a composition comprising the peptide of the present invention, or an analog, the fragment or the conjugate thereof as disclosed herein above formulated with one or more pharmaceutically acceptable excipients.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. Non-limiting examples of suitable excipients are example, water, saline, phosphate buffered saline (PBS), dextrose, glycerol, ethanol, or the like and combinations thereof. Other suitable carriers are well known to those skilled in the art. The use of such media and agents for pharmaceutically active substances is well known in the art. The compositions may contain other active compounds providing supplemental, additional, or enhanced therapeutic functions.

The peptides, analogs of fragments of the present invention could be, according to some embodiments, suspended in a sterile saline solution for therapeutic uses. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention from such a matrix depends upon the molecular weight of the molecule, the amount of the molecule within the matrix, and the size of dispersed particles.

The pharmaceutical composition of the present invention may be administered by any know method. The terms “administering” or “administration of” a substance, a compound or an agent to a subject can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A composition can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

According to some embodiments, the pharmaceutical composition is administered by an invasive mode of administration such as intramuscularly, intravenously, intra-arterially, intraarticulary or parenterally.

The administration schedule can be taken once-daily, twice-daily, thrice-daily, once-weekly, twice-weekly, thrice-weekly, once-monthly, twice-monthly, thrice-monthly, or any other administration schedule known to those of skill in the art. In addition, the administration can be continuous, i.e., every day, or intermittently. The terms “intermittent” or “intermittently” as used herein means stopping and starting at either regular or irregular intervals. For example, intermittent administration can be administration in one to six days per week or it may mean administration in cycles (e.g. daily administration for two to eight consecutive weeks, then a rest period with no administration for up to one week) or it may mean administration on alternate days.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician.

The terms “treating” of “treatment of” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

According to some embodiments, treating cancer comprises preventing or treatment tumor metastasis. According to certain embodiments, the metastasis is decreased. According to other embodiments, the metastasis is prevented.

According to some embodiments, treating cancer comprises increasing the duration of survival of a subject having cancer, comprising administering to the subject in need thereof a composition comprising a construct defined above whereby the administration of the construct increases the duration of survival.

According to some embodiments, treating cancer comprises increasing the progression of free survival of a subject having cancer.

According to some embodiments, treating cancer comprises increasing the duration of response of a subject having cancer. According to other embodiments, treating cancer comprises preventing tumor recurrence.

The cancer that may be treated according to the teaching of the present invention includes, but not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

According to some embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amendable for treatment of the invention include metastatic cancers.

According to other embodiments, the cancer is a solid cancer.

A pharmaceutically acceptable composition according to the present invention may be administered as a stand-alone treatment or in combination with a treatment by any other agent. According to a specific embodiment, constructs according to the present invention are administered to a subject in need thereof as part of a treatment regimen in combination with at least one anti-cancerous agent. The pharmaceutical composition according to the present invention may be administered in combination with another anti-cancerous agent or separately. When the composition is co-administered with another anti-cancer agent, the co-administration of the compounds is performed in a regimen selected from a single combined composition, separate individual compositions administered substantially at the same time, and separate individual compositions administered under separate schedules and include treatment regimens in which the agents are not necessarily administered by the same route of administration or at the same time. The term “co-administration” encompasses administration of a first and second agent in an essentially simultaneous manner, such as in a single dosage form, e.g., a capsule or tablet having a fixed ratio of first and second amounts, or in multiple dosage forms for each. The agents can be administered in a sequential manner in either order. When co-administration involves a separate administration of each agent, the agents are administered sufficiently close in time to have the desired effect (e.g., complex formation).

According to a specific embodiment, the anti-neoplastic (anti-cancer) composition comprises at least one chemotherapeutic agent.

The term “anti-neoplastic composition” refers to a composition useful in treating cancer comprising at least one active therapeutic agent capable of inhibiting or preventing tumor growth or function or metastasis, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegaI1 (see, e.g., Agnew, Chem Intl. Ed. Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy DNA-based vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

According to another embodiment, the present invention provides use of a peptide, analog, fragment or conjugate of the present invention in preparation of a medicament for treating cancer. According to one embodiment, the present invention provides use of a peptide comprising or consisting of SEQ ID NO: 2 in preparation of a medicament for treating cancer.

According to another aspect, the present invention provides a method of treating a cell proliferative disease. According to one embodiment, the cell proliferative disease is cancer. Thus, in some embodiment, the present invention provides a method of treating cancer in a subject in need thereof comprising administering to said subject a peptide of the present invention. According to some embodiment, the peptide comprises an amino acid sequence selected from SEQ ID NO: 2 and SEQ ID NO: 1. According to one embodiment, the method comprises administering a peptide consisting of amino acid sequence selected from SEQ ID NO: 2 or SEQ ID NO: 1. According another embodiment, the method comprises administering a conjugate of a peptide comprising or consisting of SEQ ID NO: 2. According a further embodiment, the method comprises administering a conjugate of a peptide comprising amino acid sequence SEQ ID NO: 1. According to some embodiments, the method comprises administering an analog, such as a conservative analog, of a peptide comprising amino acid sequence selected from SEQ ID NO: 2 or SEQ ID NO: 1. According to other embodiments, the method comprises administering a conjugate of a peptide comprising amino acid sequence selected from SEQ ID NO: 2 or SEQ ID NO: 1. According to any one of the above embodiments, the peptide, analog or fragment of the present invention is cyclic peptides.

According to some embodiments, the pharmaceutical composition is administered as part of a treatment regimen in combination with at least one additional anti-cancer agent.

According to some embodiment, the method comprises administering a pharmaceutically effective amount of said peptide or said conjugate. According to some embodiments, the method comprises administering a pharmaceutical composition comprising said peptide, analog thereof or said conjugate.

According to some embodiments, the pharmaceutical composition is administered as part of a treatment regimen together with at least one anti-cancer agent. The term “therapeutically effective amount” is an amount of a drug, compound, construct etc. that, when administered to a subject will have the intended therapeutic effect. The full therapeutic effect does not necessarily occur by administration of one dose, and may occur only after administration of a series of doses.

According to one aspect, the present invention provides a polynucleotide comprising a nucleic acid sequence encoding the peptide, conservative analog, analog or fragment of the present invention. According to some embodiments, the polynucleotide is an isolated polynucleotide.

The term “isolated”, when applied to a nucleic acid or protein, denotes that the nucleic acid or protein is essentially free of other cellular components with which it is associated in the natural state.

The term “nucleic acid” encompasses DNA, RNA, single stranded or double stranded and chemical modifications thereof. The terms “nucleic acid” and “polynucleotide” are used interchangeably herein.

According to one embodiment, the polynucleotide encodes a peptide comprising SEQ ID NO: 2. According to another embodiment, the polynucleotide encodes a peptide consisting of SEQ ID NO: 2. According another embodiment, the polynucleotide encodes an analog of a peptide comprising or consisting of SEQ ID NO: 2. According to yet another embodiment, the polynucleotide encodes a fragment of the peptide comprising or consisting of SEQ ID NO: 2 or of an analog thereof.

According to other embodiments, the polynucleotide encodes a peptide comprising SEQ ID NO: 1. According to another embodiment, the polynucleotide encodes a peptide consisting of SEQ ID NO: 1. According another embodiment, the polynucleotide encodes an analog of a peptide comprising or consisting of SEQ ID NO: 1. According to yet another embodiment, the polynucleotide encodes a fragment of the peptide comprising or consisting of SEQ ID NO: 1 or of an analog thereof.

According to another aspect, the present invention provides a nucleic acid construct comprising the polynucleotide of the present invention operably linked to a promoter. According to another embodiment, the polynucleotide encodes a peptide comprising amino acid sequence SEQ ID NO: 2. According to a further embodiment, the polynucleotide encodes a peptide comprising amino acid sequence SEQ ID NO: 1.

The term “nucleic acid construct” as used herein, refers to an artificially constructed segment of nucleic acid. It can be an isolate or integrated into another nucleic acid molecule.

As used herein, the term “operably linked”, “operably encodes”, and “operably associated” are used herein interchangeably and refer to the functional linkage between a promoter and nucleic acid sequence, wherein the promoter initiates transcription of RNA corresponding to the DNA sequence.

The term “promoter” is a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability.

According to another aspect, the present invention provides a vector comprising the polynucleotide or the nucleic acid construct of the present invention. According to one embodiment, the polynucleotide or the nucleic acid construct encodes a peptide comprising or consisting of SEQ ID NO: 2. According to another embodiment, the polynucleotide or the nucleic acid construct encodes a peptide comprising or consisting of SEQ ID NO: 1.

The terms “vector” and “expression vector” are used herein interchangeably and refer to any non-viral vector such as plasmid, cosmid, artificial chromosome (bacterial or yeast), or viral vector such as virus, retrovirus, bacteriophage, or phage, binary vector in double or single stranded linear or circular form, or nucleic acid, sequence which is able to transform host cells and optionally capable of replicating in a host cell. The vector may contain an optional marker suitable for use in the identification of transformed cells, e.g., tetracycline resistance or ampicillin resistance. According to one embodiment, the vector is a plasmid. According to another embodiment, the vector is a phage or bacteriophage.

The term “plasmid” refers to circular, optionally double-stranded DNA capable of inserting a foreign DNA fragment to a cell and optionally capable of autonomous replication in a given cell. Plasmids usually contain further sequences in addition to the ones, which should be expressed, like marker genes for their specific selection and in some cases sequences for their episomal replication in a target cell. In certain embodiments, the plasmid is designed for amplification and expression in bacteria. Plasmids can be engineered by standard molecular biology techniques.

According to another aspect, the present invention provides a cell comprising the polynucleotide, the nucleic acid construct or the vector of the present invention.

The term “cell” refers to any biological cell that is capable of expressing or overexpressing the polynucleotide, the nucleic acid construct or the peptide of the present invention. According to one embodiment, the cell is a bacterial cell. According to another embodiment, the cell is an eukaryotic cell. According to a further embodiment, the cell is a plant cell or algae cell. According to some embodiments, the cell of the present invention expresses or overexpresses the peptide, analog or fragment of the present invention. According to one embodiment, the cell expresses or overexpresses the peptide comprising or consisting of SEQ ID NO: 2. According to another embodiment, the cell expresses or overexpresses the peptide comprising or consisting of SEQ ID NO: 1.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

### Example 1. Identification of the Peptide of SEQ ID NO: 2

Using the technique described in WO 2007/010525, a library of cyclic peptides was generated and tested for binding to human eukaryotic elongation factor 2 (eEF2). Following an optimization process, a cyclic peptide (denoted GW2) having the amino acid sequence Cys-Ser-Ala-Arg-Trp-Gly-Pro-Ile-Met-Pro-Trp-Cys (CSARWGPIMPWC, SEQ ID NO: 2), was identified as the most potent peptide in terms of binding to eEF2 and toxicity to cells.

### Example 2. Preparation of Multi-Armed PEG Complex Loaded with Targeting Peptides and Toxins

A construct of a branched PEG molecule covalently coupled with two different cancer-targeting moieties and one or two different peptide toxins was designed and synthesized. The targeting moieties included in this construct were the cyclic peptides E13.3 (CHPGDKQEDPNCLQADK, SEQ ID NO: 3) that binds EGFR, and PD-L1-GR (CEGLPADWAAAC, SEQ ID NO: 4) that binds to PD-L1, and the toxin moieties were the cyclic peptide toxins GW (CSARWGPTMPWC, SEQ ID NO:5), TB (CRRGSRASGAHC SEQ ID NO: 6) and GW2 of the present invention (CSARWGPIMPWC, SEQ ID NO: 2).

The preparation method comprised two steps. At the first step a branched PEG containing eight arms was produced in which seven arms are coupled with a targeting peptide/toxin peptide moiety (protected peptides) and one with a Lysine residue protected with FMOC (Fmoc-Lys). At the second step eight of the peptide/toxin-PEG molecules produced in step 1 were coupled to another branched PEG molecule of eight arms to obtain a construct of multi-branched PEG coupled with 56 toxin/targeting moieties, of which 42 moieties are toxin peptides, and 14 are targeting peptides (7 copies of EGRF targeting peptide E13.3 and 7 copies of PD-L1 targeting peptide PD-L1-GR).

In more details:

### Step 1—Preparation of Branched PEG Coupled with One Type of Targeting or Toxin Moiety

2.4 μmole of a targeting peptide or 7.3 μmole of toxin peptide were dissolved in DMSO.

All peptides have only one primary amine, except for E13.3, which has 3, of which one is protected with dde, and the N-terminal is blocked with acetate residue.

5.9 mg Fmoc-Lys-OH (Novabiochem Cat. Num. 852023; MW=368.43) was dissolved in 150 μl of HCl 0.1 M, followed by addition of 650μ1 of DMSO to reach a concentration of 20 mM. 33.4 mg of 8-arm star PEG-NHS (Mw 10 KDa, Creative Biotechnologies) were dissolved in 167 μl of dioxane to reach a concentration of 20 mM.

Each of the targeting peptides solutions were mixed with 17 μl of Fmoc-Lys-OH solution and 17 μl of PEG solution.

Each one of the toxin peptides solutions were mixed with 541 of Fmoc-Lys-OH solution and 52 μl of PEG solution. Each mix was supplemented with TEA (trimethylamine) to 5%. Each solution was incubated for 15.5 hours at room temperature on a Rotamix at 30 rpm to obtain a clear solution of 8 armed PEG coupled with 7 molecules of a specific targeting peptide/toxin peptide and one arm containing a primary amine (the Fmoc protection is removed in this process to give one free primary amine on each PEG molecule).

The branched PEG-peptide molecules are denoted PEG-E13.3, PEG-PD-L1-GR, PEG-GW, PEG-GW2 and PEG-TB.

### Step 2—Construction of Multi-Branched PEG Construct Coupled to 56 Targeting/Toxin Moieties.

Three different types of constructs were prepared: PEG-E13.3-(PD-L1-GR)-(GW2); PEG-E13.3-(PD-L1-GR)-(TB+GW) and PEG-E13.3-(PD-L1-GR)-(TB+GW2). To obtain these constructs, the branched PEG-peptide solutions of PEG-E13.3 and PEG-PD-L1-GR were mixed with one of the PEG-toxin(s): (i) PEG-GW2, (ii) PEG-GW and PEG-TB or (iii) PEG-GW2 and PEG-TB, together with 20 mM PEG-NHS solution and incubated for 2 hours at room temperature on a Rotamix at 30 rpm. The stoichiometric molar ratio of PEG-NHS:PEG-E13.3:PEG-PD-L1-GR:PEG-toxin(s) (i), (ii) or (iii) was 1:1:1:6 (when two toxins are present the molar ratio between these toxins is 1:1). Following the incubation, a hydrazine (80%) was added to a final concentration of 5%. Hydrazine was used to remove the dde protecting group from the E13.3 moiety. The mixture was incubated for 2 hours at room temperature on a Rotamix at 30 rpm. The resultant constructs are a multi-branched PEG coupled with 56 targeting/toxin peptide: 7 copies of E13.3 peptide, 7 copies of PD-L1-GR peptide, and (i) 42 copies of GW2, (ii) 21 copies of GW and 21 copies of TB or (iii) 21 copies of GW2 and 21 copies of TB. At the end of the reaction, PBS was added with gentle mixing.

### Step 3—Ultrafiltration

The samples were ultrafiltrated with two additions of 20 ml PBS using Vivaspin 20 concentrator (30 K MWCO PES) to a concentration of −206 μM of loaded multi-armed PEG denoted as PEG-E13.3-(PD-L1-GR)-(GW2); PEG-E13.3-(PD-L1-GR)-(TB+GW) and PEG-E13.3-(PD-L1-GR)-(TB+GW2)

### Example 3. Effect of Construct Comprising GW2 on the Growth and Viability of A549 Cell Line

### Materials and Methods

The test constructs PEG-E13.3-(PD-L1-GR)-(GW2); PEG-E 13.3-(PD-L1-GR)-(TB+GW) and PEG-E13.3-(PD-L1-GR)-(TB+GW2) prepared as described in Example 2, were used at concentrations of 0.3, 1 and 3 μM. Phosphate Buffered Saline (PBS) was used as a negative control.

A-549 human lung tumor cells were thawed and cultivated to achieve exponentially growing cultures. Cells were collected, counted and seeded in a 96 well tissue culture plate at the following densities: A-549: 5,000 cells/well.

The plate was incubated until the next day at 37±1° C., humidified, 5±0.5% CO₂/air, to enable cells adherence to the wells.

### Treatment

At the next day following seeding, growth media were replaced with test items solutions prepared in assay medium (2% fetal bovine serum). Test Items Solutions were applied carefully (onto the sides of the well, not directly onto the cells) in volume of 200μ1/well to achieve the final concentrations as following: PEG-E13.3-(PD-L1-GR)-(GW2): 0.3, 1, and 3 μM; PEG-E13.3-(PD-L1-GR)-(TB-GW2): 0.3, 1, and 3 μM; and PEG-E13.3-(PD-L1-GR)-(GW2) 3 μM.

The plate was incubated at 37±1° C., humidified 5±0.5% CO₂/air.

After 48 hours of incubation, representative images of treated cells were taken on microscope.

### Results

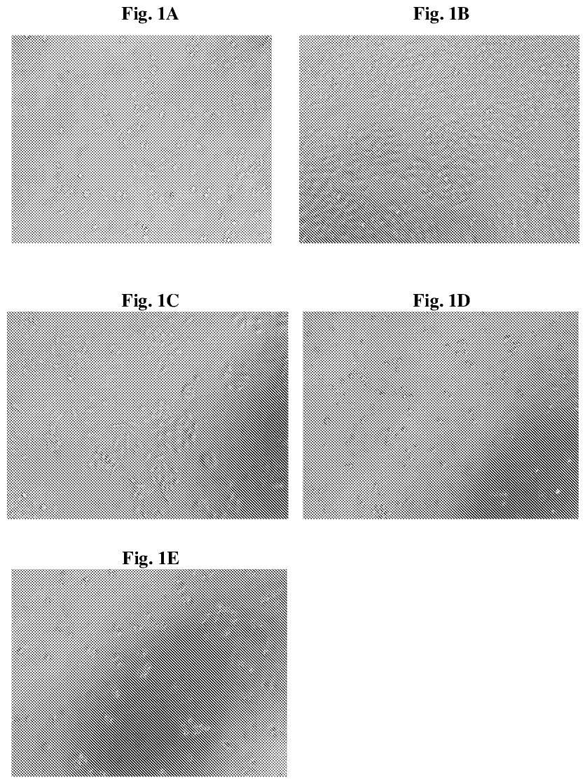

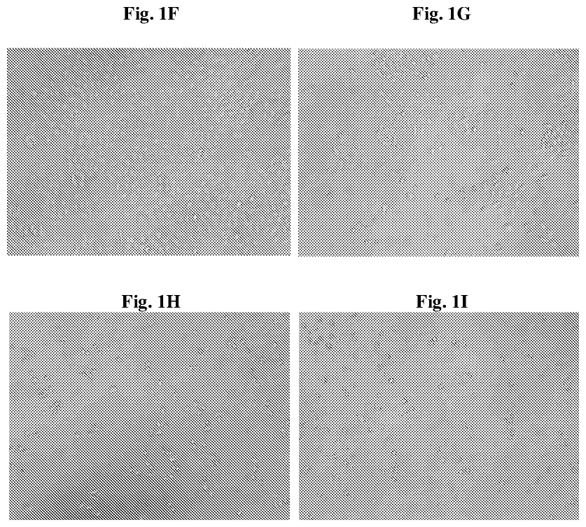

The construct comprising only GW2 (SEQ ID NO: 2) as a toxin was effective in killing A549 cell already in concentration of 0.3 μM while at concentration of 1 μM the construct complete elimination of cells was obtained. The construct comprising the mixture of GW2 (SEQ ID NO: 2) and TB (SEQ ID NO: 6) was less effective than the construct comprising GW2 alone. It may be explained by lower concentration of GW2 toxin in the construct and lower efficacy of TB toxin. The construct comprising the mixture of GW2 and TB at 3 μM achieved complete elimination of cells. Cell killing results are demonstrated in FIG. 1.

### Example 4. In Vivo Evaluation of Toxic Effect of a Multi-Armed PEG Complex Comprising GW2

### Purpose

The purpose of this study is to evaluate the potential anti-tumor activity of the test item, following 3 repeated intravenous (IV) injections, based on the relative growth inhibition of A549 human alveolar adenocarcinoma model in Athymic Nude female mice.

### Tumor Cells

Name: A549 human alveolar adenocarcinoma
Growth medium: Nutrient Mixture F-12 (Ham's), 10% fetal bovine serum, 1 mM L-Glutamine, Pen/Strep 100 u/ml, 0.1 μg/ml.
Growth conditions: 5% CO₂, 37° C.

### Cells Management

Cells were thawed and split at 1:7 to 1:10 ratios at least twice weekly.
Trypsin/EDTA was added to each flask for detachment of cells. Following detachment of cells, growth medium was added to each flask and cell suspension is vigorously pipetted to obtain a single-cell suspension.
A549 cell suspension was centrifuged for 10 minutes at 250 g.
The total number of cells and the number of dead cells was counted.
The total number of cells per ml and the viability of cells was calculated. Viability of cells must be above 90%.
The appropriate cell suspension required for injection was prepared by dilution of A549 cells in HBSS to a final concentration of 25×10⁶ cells per ml x 6 ml and placed in a vial.
After injecting all the animals the viability of the cells was evaluated in the leftover vial.

### Test Item

Construct 1—A clear solution of Construct 1 comprising a multi-armed PEG scaffold carrying E13.3, PD-L1-GR and GW2 peptides at molar ratio of 1:1:6 respectively. The construct is stored at −20° C.

### Control—PBS.

### Animals

26 Hsd:Athymic Nude-Foxn1^{nu} mice, female, 6-7 weeks of age at tumor induction, obtained from accredited breeder.

### Test Groups

Animals are assigned according to Table 1

TABLE 1

| | Tumor Induction | | Treatment | | | |
|---|---|---|---|---|---|---|
| | A549 | Route | | | Dose | Dose |
| | Concentration | & Dose | | Route & | Level | Volume |
| Group | (cells/ml) | (cells/animal) | Test Item | Regiment | (mg/kg) | (ml/kg) |
| 1F (n = 6) | 25 × 10⁶ | SC | PBS | 3 IV | 0 | 4 |
| | | 5 × 10⁶ | (Control Item) | Injection | | |
| 2F (n = 6) | | | Construct 1 | Every 2-3 | 60 | |
| 3F (n = 6) | | | | days | 180 | |

### Tumor Induction

The tumor cell suspension is injected to 26 animals at dose volume of 0.2 ml/An. (5×10⁶ cells/An.) by a single SC injection into the right flank area, midway between the axillary and inguinal regions, using 1 ml syringes and 25G needles where withdrawal is done without the needle.

### Administration Schedule

The Test Item and the Control Item were injected to n=6 tumor-bearing mice (initial tumor size of ˜100-150 mm³) per group by the IV route, at dose volume of 4 ml/kg, according to allocation Table 1. Test materials are injected 3 times (on weekdays only) during a period of a week (every 2-3 days).

### Serial Observations

All animals were observed daily for a total duration of up to 6 weeks for the following parameters.

Body Weight

Determination of individual body weights of animals is made shortly before tumor induction (Day 0) and twice weekly thereafter. In case of decedents, body weight is determined as close as possible to the time of death.

Tumor Size Follow-Up

Monitoring of progressive changes in tumor growth is carried out in all animals twice a week from measureable tumors until study termination, using Electronic Digital Calipers, the measurements is performed by the same technician, if possible. The tumor volume is determined and calculated according to the following equation:

V(mm³)=d²(mm²)×D(mm)/2

The symbols d and D represent the smallest and the largest perpendicular tumor diameters, respectively.

Although the present invention has been described herein above by way of preferred embodiments thereof, it can be modified, without departing from the spirit and nature of the subject invention as defined in the appended claims.

1-40. (canceled)

41. A peptide comprising an amino acid sequence selected from SEQ ID NO: 1 and SEQ ID NO: 2, or an analog thereof, or a fragment of the peptide or analog, wherein the peptide consists of 10 to 30 amino acids and the fragment comprises from 7 to 11 consecutive amino acids of the peptide or analog.

42. The peptide according to claim 41, wherein the peptide consists of an amino acid sequence selected from SEQ ID NO: 1 and SEQ ID NO: 2.

43. The peptide analog of claim 41, wherein the analog comprises from 1 to 4 modifications selected from the addition of an amino acid residue, deletion of an amino acid residue, substitution of an amino acid residue, modification of a terminus of the peptide, and cyclization, wherein substitution of an amino acid residue is other than substitution of an Isoleucine (Ile) residue with a Threonine (Thr) residue.

44. The peptide analog according to claim 41, wherein the peptide analog comprises from 1 to 4 conservative substitutions of amino acid residues within a sequence selected from SEQ ID NO: 1 and SEQ ID NO: 2 and wherein the peptide analog has at least 70% sequence identity to said peptide.

45. The peptide analog according to claim 44, comprising 1, 2, 3, or 4 conservative substitutions of amino acid residues within SEQ ID NO: 2.

46. The peptide, analog or fragment according to claim 41, wherein the peptide, analog or fragment is cyclic.

47. The peptide, analog or fragment according claim 41, wherein said peptide, analog or fragment specifically binds to human eukaryotic Elongation Factor 2 (eEF2), optionally wherein said binding comprises enhancing eEF2 activity.

48. A conjugate comprising at least one copy of the peptide, analog or fragment according to claim 41.

49. The conjugate according to claim 48, comprising at least one copy of a cyclic peptide comprising or consisting of SEQ ID NO: 2 and at least one cell-targeting molecule.

50. The conjugate according to claim 49, wherein the cell-targeting molecule binds specifically to a cell surface receptor selected from EGFR, PD-L1, HER2, PD-1, PD-L2, VEGFR, androgen receptor, benzodiazepine receptor, cadherin, CXCR4, CTLA-4, CD2, CD19, endothelin receptor, ERBB4, FGFR, folate receptor, HER4, HGFR, Mucin 1, OGFR, and PDGFR.

51. The conjugate according to claim 50, wherein the at least one copy of a cyclic peptide and the cancer-targeting molecule are bound directly or via a spacer or carrier.

52. The conjugate according to claim 51, comprising a plurality of cyclic peptides comprising SEQ ID NO: 2, a plurality of cell-targeting molecules that bind specifically EGFR and a plurality of cancer-targeting molecules that bind specifically PD-L1, optionally further comprising an additional toxin molecule.

53. The conjugate according to claim 52, wherein the anti-EGFR cell-targeting molecules are peptides comprising amino acid sequence SEQ ID NO: 3 and/or the anti-PD-L1 cell-targeting molecules are peptides comprising amino acid sequence SEQ ID NO: 4.

54. The conjugate according to claim 51, wherein the carrier is an organic scaffold.

55. A pharmaceutical composition comprising the peptide, analog or fragment according to claim 41 or the conjugate thereof, and a pharmaceutically acceptable carrier.

56. The pharmaceutical composition according to claim 55, comprising a cyclic peptide comprising amino acid sequence SEQ ID NO: 2 or a conjugate of the cyclic peptide.

57. A method of treating cancer in a subject in need thereof comprising administering to said subject a pharmaceutically effective amount of the peptide, analog or fragment according to claim 41, a conjugate thereof, or a pharmaceutical composition comprising said peptide, analog, fragment, or conjugate and a pharmaceutically acceptable carrier.

58. The method according to claim 57, wherein the pharmaceutical composition is administered in combination with at least one additional anti-cancer agent.

59. An isolated polynucleotide, a nucleic acid construct or a vector comprising a nucleic acid sequence encoding the peptide, analog or fragment according to claim 41.

60. A cell comprising the polynucleotide, nucleic acid construct or vector according to claim 59.
